(19) United States
(12) Patent Application Publication
(10) Pub. No.: US 2009/0299305 A1
(43) Pub. Date: Dec. 3, 2009

(54) COLLECTION SYSTEM FOR BIOLOGICAL SAMPLE

(76) Inventor: Albert Anouna, Farhills, NJ (US)

Correspondence Address: Pearl Cohen Zedek Latzer, LLP, 1500 Broadway, 12th Floor, New York, NY, 10036, US

(21) Appl. No.: 12/279,524

(22) PCT Filed: Feb. 12, 2007
(86) PCT No.: PCT/US07/03789

§ 371 Date: Jan. 14, 2009

Related U.S. Application Data
(60) Provisional application No. 60/772,904, filed on Feb. 14, 2006.

(51) Int. Cl. A61M 1/00

(52) U.S. Cl. 604/317

(57) ABSTRACT

This invention relates to a system for collecting and preserving biological sample. Specifically, the invention relates to an assembly for collecting, preserving and transporting biological samples, methods of using the assembly and kits comprising the same.

### FIELD OF INVENTION

This invention is directed to a system for collecting and preserving biological sample. Specifically, the invention relates to an assembly for collecting, preserving and transporting biological samples, methods of using the assembly and kits comprising the same.

### BACKGROUND OF THE INVENTION

The analysis of freshly ejaculated semen is the most important diagnostic tool in the initial investigation of male fertility and the proper assessment of semen quality is essential in the diagnosis of several treatable disorders of male fertility. For the semen analysis result to be most valuable, proper collection of the specimen is essential.

The specimen is best collected by masturbation into a sterile container. This is usually performed in a facility provided at the laboratory, however collection at home is acceptable provided the sample is rapidly transported and kept at body temperature or preferably at 1° C. lower. Semen collected by interrupted intercourse is not favored as it risks the loss of sample, particularly the first fraction of the ejaculate. Moreover, semen should never be collected into an ordinary condom, which contains substances that kill sperm. In situations where religious or personal practices prohibit masturbation, or psychological inhibitions interfere, a special condom (SCD) may be used that does not affect the sperm quality, so long as the semen is immediately transported into an acceptable receptacle.

There is a need for a collection system for biological fluids, which is easy to operate, requiring minimal manipulation of the collected specimen, while maintaining the integrity of the biological sample ensuring safe and efficient transportability without the risk of spoilage tampering and contamination.

### SUMMARY OF THE INVENTION

In one embodiment, the invention provides an assembly for collecting a biological sample comprising: a flared cylinder base having a narrow top end and a wide bottom end, the base capable of housing a removable tube therein; a removable tube, wherein the removable tube is capable of being hermetically sealed and fits into the flared cylinder base; a collection funnel attached to the narrow top end of said flared cylinder base; and a snap cap, attached to said funnel, wherein said collection funnel allows the biological sample to gravimetrically flow directly into the removable tube, thereby being an assembly for the collection of a biological sample.

In another embodiment, the invention provides a method for collecting a biological sample comprising the steps of: opening a snap-cap attached to the top of a collection funnel, which is attached to the narrow end of a flared cylinder base capable of housing a removable tube therein; dispensing a biological sample into the collection funnel; allowing the biological sample to gravimetrically flow into a removable tube disposed within the flared cylinder base; and sealing the removable tube, thereby collecting a biological sample.

In one embodiment, the invention provides a kit for the collection of a biological sample, comprising packaging materials; the assembly for the collection of a biological sample comprising: a flared cylinder base having a narrow top end and a wide bottom end, capable of housing a removable tube therein; a collection funnel attached to the narrow end of said base; a removable tube and a snap cap, hingedly attached to said funnel; labels; and instructions.

### BRIEF DESCRIPTION OF THE DRAWINGS

FIG. 1 shows a schematic operation of an embodiment of the kit of the invention; and

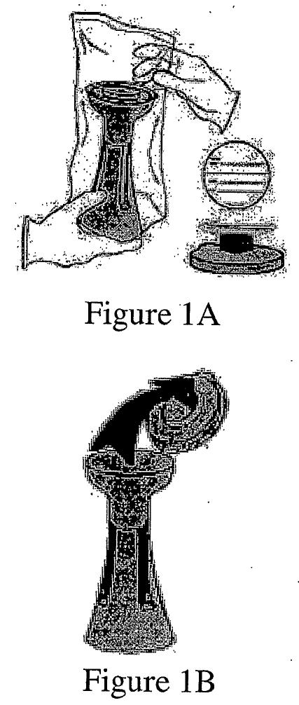

FIG. 2 shows a sample of the graduated tube housed in the sample collection assembly.

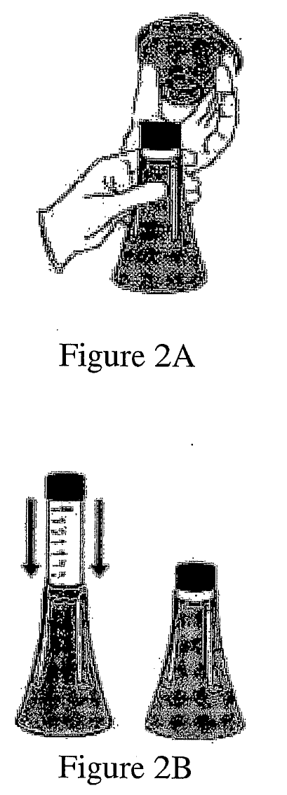

### DETAILED DESCRIPTION OF THE INVENTION

This invention relates in one embodiment to an assembly for the collection of biological samples, method for its use and a kit comprising the same.

In one embodiment, the systems, methods and kits described herein are used for collecting, carrying and processing biological samples by clients such as a subject wishing to collect semen for the purpose of testing fertility. In another embodiment, collecting the semen specimen is desirable for testing viability and motility of sperm. In another embodiment, the semen collection is done for the purpose of donating sperm to a donee, such as another individual or a sperm-bank. In one embodiment the collected semen can be safely utilized in an “In vitro” or Assisted Reproduction Technique (ART) to create an embryo for implantation. In one embodiment, the assembly is used for the collection of semen, or saliva, blood, plasma, sputum or mucus in other embodiments.

In one embodiment, the invention provides an assembly for the collection of a biological sample comprising: a flared cylinder base having a narrow top end and a wide bottom end, the base capable of housing a removable tube therein; a removable tube, wherein the removable tube is capable of being hermetically sealed and fits into the flared cylinder base; a collection funnel attached to the narrow top end of said flared cylinder base; and a snap cap, hingedly attached to said funnel, wherein said collection funnel allows the biological sample to gravimetrically flow directly into the removable tube, thereby being an assembly for the collection of a biological sample.

In one embodiment, the assembly for the collection of a biological sample is used by laboratory personnel in collecting, handling, carrying and further processing biological samples.

In one embodiment, the removable tube used in the assemblies, methods and kits described herein, is a graduated tube. In one embodiment, the term graduated tube refers to a tube having visible marks and numbers at vertical intervals, permitting one to estimate the quantity of material contained in the vessel, most commonly those used in laboratories for containing liquids. In one embodiment, the quantity estimated using the gradations on the tube is the volume of semen obtained. In one embodiment, the graduated tube used, has a 20 ml volume with graduation at 2 ml intervals, or 1 ml intervals or 0.5 ml intervals in other embodiments. In one embodiment the gradations allow precise evaluation of the contained volume to within about ±0.25 ml of the actual volume.

In one embodiment, the removable tube used is a centrifuge tube, capable of withstanding 10,000·g. The tube used is in one embodiment chemically resistant to alcohols and mild organic solvents with a hydrophobic, biologically inert surface that can withstand temperatures from about −80 to about 120° C. In one embodiment, the term “10,000·g”, or “g”, refers to the relative centrifugal force exerted by the centrifuge on the contents of the removable tube as described herein. In one embodiment, all surfaces which may come in contact with the semen specimen are biologically inert, referring to these surfaces being free of heavy metals, color concentrates, DNAase, RNAase or similar proteinases or other substances which, when in contact with the semen specimen, may alter its properties so as to render further analysis erroneous.

In one embodiment, the centrifugal force is dependent upon the radius of the rotation of the rotor, the speed at which it rotates, and the design of the rotor itself (e.g. fixed angle, or swinging bucket). Rotor speed and design can be held constant, but the radius will vary from the top of a centrifuge tube to the bottom and will be dependent in one embodiment on the dimensions of the tube. If a measurement for the radius is taken as the mid-point, or as an average radius, and all forces are mathematically related to gravity, then one obtains a relative centrifugal force, labeled as xg. Centrifugation of semen samples are given in one embodiment as xg measures, since RPM and other parameters will vary with the particular instrument and rotor used. Relative Centrifugal Force refers in another embodiment to a constant that is independent of the apparatus used. In one embodiment, the removable tube used in the systems, methods and kits described herein, is capable of withstanding RCF up to 10,000·g.

In one embodiment, all surfaces which may come in contact with the semen specimen are biologically inert and will not allow the specimen to wet the surfaces. In one embodiment, the term “wet” or “wetting”, as used herein, refers to the wettability of the membrane by the semen sample with which it is contacted. The wettability of a solid structure, e.g., a collection funnel, is a function of that structure's critical surface energy and the surface tension of the applied liquid. When the critical surface energy is substantially lower than the surface tension of the liquid, the liquid will spontaneously bead and form droplets on the solid structure. In one embodiment, a collection funnel having a critical surface energy of about 18-36 dynes/cm or lower will not be wetted by a semen specimen which has a surface tension higher than 36 dynes/cm, i.e., the collection funnel is hydrophobic. In one embodiment, the hydrophobic nature of the contact surfaces facilitates better gravimetric flow of the specimen into the removable tube and from the removable tube upon further processing.

In one embodiment, the removable tube, or the collection funnel or both are made of glass, or poly(propylene) (PP) which may be clarified in one embodiment, poly(carbonate) (PC), poly(methacrylate) (PMA), or Poly(ethylenetheraphtalate) (PET) including CPET and APET, Polypropene, Polipropene25 [USAN™], propene polymers, Propylene polymers, 1-propene homopolymer, poly(1-methyethelyne), or a combination thereof in other embodiments.

In one embodiment, sealing the removable tube used in the systems, methods and kits with the screw-cap described herein, creates a tamper evident package. The term “tamper evident package” refers to packaging having an indicator or barrier to entry which, if breached or missing, can reasonably be expected to provide visible or audible evidence to end-users that tampering has occurred. Tamper-evident packaging may involve immediate-vessel/packaging systems or any combination thereof. It provides in one embodiment, a visual indication of package integrity when handled in a reasonable manner during manufacture, distribution and retail and in-process supply. In one embodiment, the visual indication is accompanied by appropriate precautionary label statements to describe the tamper-evident feature(s) to the user and to warn that the absence of, or damage to such feature(s) at the time of purchase or once in the laboratory processing the semen specimen, is an indication of possible tampering with the product.

In one embodiment, the kits described herein, which comprise the systems described herein, are contained in a tamper evident packaging. In one embodiment, the components of the assembly for the collection of biological samples, are enclosed in an individual pouch that must be torn or broken to obtain its contents. The pouch has in another embodiment a distinctive design (e.g., a pattern, name, registered trademark, logo, or picture).

In one embodiment, the screw cap used to seal the removable tube used in the systems described herein, has a flexible bead system that provides increased gripping to break the tamper-evident webs. The folding-bead linerless seal doesn't require an extra liner to remain closed, making it cost effective. In another embodiment, a foam liner inside the cap is disposed such that a pressure sensitive adhesive makes the liner adhere to the neck of the removable tube upon closure by the user, making the liner a tamper evidence means.

In one embodiment, the flared cylinder base defines at least one observation window allowing observation of the removable tube disposed therein. The observation window may be an opening in the continuous base wall, which is opaque in one embodiment. In another embodiment, the window defined by the opening in the base, is covered by a translucent material, which is made of a material that is either the same or different than the material the base is made of. In one embodiment, three windows are defined by axial opening in the base, and guides within the narrow end of the base, which correspond to guides disposed on the removable tube, align the removable tube such that one observation window allows the observation of the sample inside the removable tube, while the other window allows the observation of a label disposed at a fixed location on the removable tube. In one embodiment, the flared cylinder base used in the systems described herein, is made of High-density poly(ethylene) (HDPE), while in another embodiment, the window or windows defined therein, are covered by clarified PP, or PC, cPET or PMMA in other embodiments.

In one embodiment, the based is a flared cylinder having a bottom end and a top end, wherein the ratio between the diameter of the bottom end and the top end is no less than 1.25. In one embodiment, the based has a flared cylinder shape, with the wider diameter being at the bottom and wherein the ratio between the wide end and narrow end of the flared cylinder base, is no less than 1.25. In one embodiment, the diameter of the open end of the collection funnel is the same as the diameter of the bottom end of the base.

In one embodiment, the base is made of a translucent material, obviating the need for an observation window. In another embodiment, the base is made of clarified PP and has no windows disposed thereon.

In one embodiment, the collection funnel used in the systems, methods and kits described herein, has a snap-cap hingedly connected to the body of the collection funnel and disposed thereon. In one embodiment, the snap cap has a tamper evident band frangibly connected to the body portion of the collection funnel, wherein the tamper evident band is shaped, sized and positioned to engage structure on the snap-cap portion of the assembly, in order to preclude movement of the snap-cap away from the closed position, wherein the structure on the snap-cap comprises a projection extending radially outwardly from the snap-cap portion, and where the projection is shaped, sized and positioned to engage an underside of said tamper evident band.

In one embodiment, the snap-cap or flip-top cap is integrally attached to the collection funnel. In one embodiment, the collection funnel has an upper portion and an inner and outer surface, wherein the collection funnel has a cylindrical upstanding rim at the upper portion, the rim is defined as that portion of the collection funnel that contacts an inner wall of the snap-cap or flip-top cap, the rim having an inner and outer wall rim, whereby the inner rim wall is substantially parallel to the inner surface of the collection funnel.

In one embodiment, the snap-cap or flip-top cap consists of one hinge attached to the collection funnel wherein the hinge has at least one hinge recess bend point that functions to rotate the snap-cap or flip-top cap at one pivot point, the thumb tab and the hinge being positioned on substantially opposing ends of the snap-cap or flip-top cap and extending perpendicularly and outwardly from the skirt of the snap-cap or flip-top cap.

In one embodiments, the methods described herein, use the assemblies for the collection of biological sample described hereinabove.

In one embodiment, the invention provides a method for collecting a biological sample comprising the steps of: opening a snap-cap hingedly attached to the top of a collection funnel, wherein the collection funnel is attached to the narrow end of a flared cylinder base capable of housing a removable tube therein; dispensing a biological sample into the collection funnel; allowing the biological sample to gravimetrically flow into a removable tube disposed within the flared cylinder base; and sealing the removable tube, thereby collecting a biological sample.

In one embodiment, the step of sealing the removable tube comprises the step of removing the collection funnel from the base; and sealing the removable tube with a screw-cap capable of hermetically sealing the removable tube, wherein the screw cap has a pressure sensitive liner inside the cap, whereupon closing of the removable tube, the liner adheres to the removable tube's body, thereby becoming a tamper evidence means.

In another embodiment, the step of sealing the removable tube comprises closing the snap-cap hingedly attached to the body of the collection funnel. In one embodiment, closing the snap-cap further involves the step of attaching a label to the snap-cap such that the label traverses the snap-cap body and attaches to both the snap cap and the collection funnel body, thereby becoming a tamper evidence means.

In one embodiment, the methods, assemblies and kits described herein are used to collect biological samples from a subject. The biological sample to be collected may consist in one embodiment of, or comprise blood, sera, urine, mucosa, sputum feces, epidermal sample, skin sample, cheek swab, sperm, amniotic fluid, cultured cells, bone marrow sample or chorionic villi, and the like.

In one embodiment, the kits of the invention described herein, are used to carry out the methods described hereinabove. In one embodiment, the invention provides a kit for the collection of a biological sample, comprising packaging materials; the assembly for the collection of a biological sample comprising: a flared cylinder base having a narrow top end and a wide bottom end, capable of housing a removable tube therein; a collection funnel attached to the narrow end of said base; a removable tube and a snap cap, hingedly attached to said funnel; labels; and instructions.

In another embodiment, the kits of the invention may be adjusted for a specific biological sample. In one embodiment, when the desired biological sample to be collected is semen, the kit may further comprise a condom. In one embodiment, the kits described herein may comprise additional labels or chemicals to facilitate the collection, transportation or analysis of the collected sample.

In one embodiment, some components may be supplied separately from the kit, or obtained by the end-user separately from the kit. In one embodiment, the removable tube in the assembly may be a 20 ml graduated centrifuge tube, having a conical bottom, which in one embodiment may be purchased separately from the kit as described herein.

In one embodiment, the labels provided as part of the kits as described herein, may be the same and may be affixed on the various components of the kit, including in another embodiment, the discrete parts of the assembly, the removable tube, the screw-cap, the snap-cap or flip-top cap and the like.

The term “about” as used herein means in quantitative terms plus or minus 5%, or in another embodiment plus or minus 10%, or in another embodiment plus or minus 15%, or in another embodiment plus or minus 20%.

The foregoing has been a description of certain non-limiting embodiments of the invention, including preferred embodiments. Those of ordinary skill in the art will appreciate that various changes and modifications to this description may be made without departing from the spirit or scope of the present invention, as defined in the following claims.

What is claimed is:

1. An assembly for collecting a biological sample comprising: a flared cylinder base having a narrow top end and a wide bottom end, the base capable of housing a removable tube therein; a removable tube, wherein the removable tube is capable of being hermetically sealed and fits into the flared cylinder base; a collection funnel attached to the narrow top end of said flared cylinder base; and a snap cap, attached to said funnel, wherein said collection funnel allows the biological sample to gravimetrically flow directly into the removable tube, thereby being an assembly for the collection of a biological sample.

2. The assembly of claim 1, wherein said biological sample is blood, sputum, sera, urine, mucosa, feces, epidermal sample, skin sample, cheek swab, sperm, semen, amniotic fluid, cultured cells, bone marrow sample, chorionic villi or a combination thereof.

3. The assembly of claim 1, wherein the ratio between the diameter of the wide bottom end of the flared cylinder base and the diameter of the narrow top end of the flared cylinder base is about 1.25.

4. The assembly of claim 1, wherein said removable tube is graduated.

5. The assembly of claim 1, wherein said removable tube is capable of withstanding relative centrifugal force (RCF) of up to about 10,000·g

6. The assembly of claim 1, wherein said flared cylinder base comprises at least one observation window, axially disposed thereon.

7. The assembly of claim 4, wherein said graduated removable tube is a removable 15 ml graduated conical tube.

8. The assembly of claim 1, further comprising a screw-cap, wherein said screw-cap is capable of sealing the removable tube hermetically.

9. The assembly of claim 8, wherein the screw-cap comprises tamper evidence means.

10. The assembly of claim 1, wherein the removable tube is made of a material that is inert to the biological sample.

11. The assembly of claim 10, wherein said inert material is glass, poly(propylene) (PP), poly(carbonate) (PC), poly(methacrylate) (PMA), or Poly(ethylenetheraphtalate) (PET) including CPET, APET, Polypropene, Polipropene25 [USAN™], propene polymers, Propylene polymers, 1-propene homopolymer, poly(1-methyethelyne), or a combination thereof.

12. The assembly of claim 1, wherein the collection funnel is made of glass, poly(propylene) (PP), poly(carbonate) (IC), poly(methacrylate) (PMA), or Poly(ethylenetheraphtalate) (PET) including CPET and APET.

13. The assembly of claim 10, wherein said inert material is substantially free of heavy metals or color concentrates.

14. The assembly of claim 12, wherein said collection funnel is made of a material which is substantially free of heavy metals or color concentrates

15. The assembly of claim 1, wherein the flared cylinder base is made of High-density poly(ethylene) (HDPE)

16. A method for collecting a biological sample comprising the steps of: opening a snap-cap attached to the top of a collection funnel, which is attached to the narrow end of a flared cylinder base capable of housing a removable tube therein; dispensing a biological sample into the collection funnel; allowing the biological sample to gravimetrically flow into a removable tube disposed within the flared cylinder base; and sealing the removable tube, thereby collecting a biological sample.

17. The method of claim 16, wherein sealing the removable tube comprise the steps of: removing the collection funnel from the base; and sealing the removable tube with a screw-cap capable of hermetically sealing the removable tube.

18. The method of claim 16, wherein sealing the removable tube comprise the steps of: closing the snap cap at the top of the collection funnel.

19. The method of claim 16, wherein said biological sample is blood, sera, urine, mucosa, feces, epidermal sample, skin sample, cheek swab, sperm, semen, amniotic fluid, cultured cells, bone marrow sample, chorionic villi or a combination thereof.

20. The method of claim 16, wherein said removable tube is graduated.

21. The method of claim 16, wherein said removable tube is capable of withstanding relative centrifugal force (rcf) of up to about 10,000·g

22. The method of claim 16, wherein said flared cylinder base comprises at least one observation window, axially disposed thereon.

23. The method of claim 20, wherein said graduated removable tube is a removable 20 ml graduated conical tube.

24. The method of claim 16, wherein the screw-cap capable of hermetically sealing the removable tube further comprises tamper evidence means.

25. A kit for the collection of a biological sample, comprising: packaging materials; the assembly of claim 1; a screw-cap; a label; and instructions.

26. The kit of claim 25, wherein said biological sample is blood, sera, urine, sputum, mucosa, feces, epidermal sample, skin sample, cheek swab, sperm, semen, amniotic fluid, cultured cells, bone marrow sample or chorionic villi.

27. The kit of claim 26, wherein the biological sample is semen.

28. The kit of claim 27, further comprising a condom.
